(19) United States
(12) Patent Application Publication
(10) Pub. No.: US 2022/0061831 A1
(43) Pub. Date: Mar. 3, 2022

(54) APPROXIMATING ANCHOR DEVICE

(71) Applicants: The Texas A&M University System, College Station, TX (US); Baylor College of Medicine, Houston, TX (US); Fannin Partners, LLC, Houston, TX (US)

(72) Inventors: Achu Geetha Byju, College Station, TX (US); Balakrishna Haridas, College Station, TX (US); Jimmy Espinoza, Houston, TX (US); Michael J. Belfort, Houston, TX (US); Michael J. Heffernan, Katy, TX (US)

(73) Assignees: The Texas A&M University System, College Station, TX (US); Baylor College of Medicine, Houston, TX (US); Fannin Partners, LLC, Houston, TX (US)

(21) Appl. No.: 17/420,212

(22) PCT Filed: Jan. 3, 2020
(86) PCT No.: PCT/US2020/012129

§ 371 Date: Jul. 1, 2021

Related U.S. Application Data
(60) Provisional application No. 62/788,664, filed on Jan. 4, 2019.

(51) Int. Cl. A61B 17/04; A61B 1/313

(52) U.S. Cl. CPC A61B 17/0401; A61B 2017/4216; A61B 1/3132

(57) ABSTRACT

An approximating anchor device, corresponding kits, and methods for implementing the device are described herein. In one embodiment, the approximating anchor device includes a first cylindrical fastener defining a first set of cross lumens, each of the first set of cross lumens extending substantially perpendicular to a central axis of the first cylindrical fastener, a second cylindrical fastener defining a second set of cross lumens, each of the second set of cross lumens extending substantially perpendicular to a central axis of the second cylindrical fastener, and a string running through the first set of cross lumens of the first cylindrical fastener and the second set of cross lumens of the second cylindrical fastener. Tightening of the string will draw the first cylindrical fastener and the second cylindrical fastener towards each other.

### CROSS-REFERENCE TO RELATED APPLICATION

This application claims the benefit of priority to U.S. Provisional Patent Application Ser. No. 62/788,664, filed Jan. 4, 2019. The entire content of this application is hereby incorporated by reference herein.

### STATEMENT REGARDING FEDERALLY SPONSORED RESEARCH OR DEVELOPMENT

This invention was made with government support under Grant No. SBIR Phase 1 R43 HD094456-01 awarded by the National Institutes of Health. The government has certain rights in the invention.

### BACKGROUND OF THE INVENTION

Multiple surgical situations call for the apposition, approximation, and fixation of two or more layers of tissue without the luxury of direct visualization. Examples include laparoscopic abdominal surgery (e.g., colo-rectal, bariatric, etc.), gastropexy (e.g., attaching the stomach to the abdominal wall), endoscopy (e.g., colonoscopy, GI endoscopy, etc.), cardiothoracic surgery, urogynecologic surgery, and others.

One specific example of these situations occurs when there is a need to approximate the uterine (e.g., the chorioamniotic) membranes to the uterine wall in fetal surgery under ultrasound guidance. Conditions such as spina bifida may be treated through open fetal surgery or performed through ports placed in the uterus, which may be generally referred to as fetoscopic surgery. Numerous other congenital fetal abnormalities exist that may benefit from fetoscopic surgery, such as congenital diaphragmatic hernia (CDH), critical aortic valve stenosis, twin-twin transfusion syndrome (TTTS), fetal lower urinary tract obstruction etc.

In order to perform fetoscopic surgeries, a trocar or port is often implanted through the abdomen, through the wall of the uterus, and through the placental membrane. However, these insertions may cause defects in the uterine membranes and may lead to separation of the chorion and the amnion or rupturing of the placental membranes (e.g., preterm premature rupture of membranes), which may in turn lead to premature delivery of the baby.

To reduce the risk of premature rupture of membranes, the conventional approach in fetal surgery is to place sutures to “anchor” the placental membrane to the uterine wall. However, this requires an incision in the maternal abdomen (e.g., a laparotomy) to exteriorize the uterus. Thus, anchoring the chorioamniotic membranes in some cases cannot be accomplished when the fetal surgery is performed using a percutaneous approach (e.g., without laparotomy and uterine exteriorization). In addition, when the uterus is exteriorized, the sutures are placed under ultrasound guidance using curved needles; however, the ultrasonographic visualization of a curved needle to place a suture in the uterus is frequently challenging. The conventional approaches for some fetal surgeries are highly invasive surgery because they require laparotomy and manipulation of the uterus, which typically requires longer recovery time post-surgery.

### SUMMARY OF THE INVENTION

One aspect of the invention provides a fastener assembly for securing a first bodily wall to a second bodily wall. The fastener assembly includes: a first cylindrical fastener defining a first set of cross lumens, each of the first set of cross lumens extending substantially perpendicular to a central axis of the first cylindrical fastener; a second cylindrical fastener defining a second set of cross lumens, each of the second set of cross lumens extending substantially perpendicular to a central axis of the second cylindrical fastener; and a string running through the first set of cross lumens of the first cylindrical fastener and the second set of cross lumens of the second cylindrical fastener, such that the tightening of the string will draw the first cylindrical fastener and the second cylindrical fastener towards each other.

This aspect of the invention can have a variety of embodiments. The string can be fastened to itself with a locking-sliding knot. The string can be fastened to itself with a Weston knot or a Meltzer knot.

The first cylindrical fastener and the second cylindrical fastener can each have a maximum cross-sectional dimension of about 2.5 mm.

The first cylindrical fastener, second cylindrical fastener, and the string can comprise a resorbable polymer composition. The resorbable polymer composition can comprise one or more materials selected from the group consisting of: polyglycolide-lactide copolymers, polyglactin 910, polydioxanone (PDS), polyglycolic acid, polylactic acid, polycaprolactone, and polylactide-caprolacrone copolymers. The first cylindrical fastener, the second cylindrical fastener, the string, of a combination thereof, can comprise one or more materials selected from the group of: thermoplastic bioresorbable polyurethanes with degradable hard and soft segments, synthesized from polyester polyols, and aromatic or aliphatic isocyanates. The design of the cylindrical fastener and string, and the polymer composition can be engineered to provide a target degradation time.

The first cylindrical fastener can further define a recess located along a longitudinal axis of the first cylindrical fastener. A portion of the string can rest in the recess when the longitudinal axis of the first cylindrical fastener is perpendicular to the first bodily wall, the second bodily wall, or both. The longitudinal axis of the first cylindrical fastener can reposition from being substantially perpendicular to the first bodily wall, the second bodily wall, or both, to being substantially parallel to the first bodily wall, the second bodily wall, or both, during the tightening of the string.

Another aspect of the invention provides an anchor delivery or deployment device including: a shaft defining a longitudinal cavity, the longitudinal cavity having a set of dimensions to fit the fastener assembly as described herein within the longitudinal cavity.

This aspect of the invention can have a variety of embodiments. The anchor delivery or deployment device can further include an obturator having a piercing end, the obturator having a width to allow the obturator to fit within the longitudinal cavity of the shaft. The obturator can terminate in a handle opposite the piercing end. The shaft can further include a cannula.

Another aspect of the invention provides a method of securing a first bodily wall to a second bodily wall of a subject. The method includes: inserting the shaft of the anchor device as described herein into the skin of the subject, through the first bodily wall and through the second bodily wall; inserting the fastener assembly into the shaft of the anchor device; deploying the first cylindrical fastener inside of the second bodily wall; withdrawing the shaft of the anchoring device through the second bodily wall and the first bodily wall; deploying the second cylindrical fastener from the shaft of the anchoring device and outside of the first bodily wall; cinching the first cylindrical fastener and the second cylindrical fastener together by pulling one or both ends of the string; and withdrawing the shaft of the anchoring device completely from the skin of the subject.

This aspect of the invention can have a variety of embodiments. The method can further include inserting an obturator into the skin of the subject to define an opening through the first bodily wall and the second bodily wall, wherein inserting the shaft of the anchor device further comprises inserting the shaft of the anchor device into the opening. Insertion and manipulation of the obturator can be aided by medical imaging. The medical imaging can be ultrasound or direct visualization using a laparoscope or fetoscope.

The first bodily wall can be a uterine wall and the second bodily wall can be a placental membrane. The subject can be a mammal. The subject can be a human. The subject can be a pregnant female.

Another aspect of the invention provides a kit including: a fastener assembly; and instructions to perform a method described herein.

Another aspect of the invention provides a fastener assembly for securing a first bodily wall to a second bodily wall. The fastener assembly includes: a first cylindrical fastener; a second cylindrical fastener; and a string. The first cylindrical fastener defines: a first axial external channel; a second axial external channel; a first cross lumen extending between the first axial external channel and the second axial external channel; and a second cross lumen extending between the first axial external channel and the second axial external channel. The second cylindrical fastener defines: a third axial external channel; a fourth axial external channel; a third cross lumen extending between the third axial external channel and the fourth axial external channel; and a fourth cross lumen extending between the third axial external channel and the fourth axial external channel. The string runs through the first cross lumen and the second cross lumen of the first cylindrical fastener, and the third cross lumen and the fourth cross lumen of the second cylindrical fastener, such that the tightening of the string will draw the first cylindrical fastener and the second cylindrical fastener towards each other.

Another aspect of the invention provides a fastener system comprising: a delivery cylinder comprising: a proximal end adapted and configured for manipulation by a user; and a distal end adapted and configured for advancement through one or more bodily walls; a fastener assembly as described herein; and a pusher. The pusher includes: a proximal end adapted and configured for manipulation by a user; and a distal end adapted and configured for advancement into the delivery chamber, thereby advancing the fastener assembly. The proximal end of the delivery cylinder and the proximal end of the pusher have complimentary mechanical logic that: limits initial advancement of the pusher rod to initially advance only a distal fastener beyond the distal end of the delivery cylinder; and permits further advancement of the pusher in response to a second action to advance a proximal beyond the distal end of the delivery cylinder.

This aspect of the invention can have a variety of embodiments. The pusher can define an axial channel extending between the proximal end and the distal end, the axial channel adapted and configured to receive the string. The axial channel can be internal or external to the pusher.

The complimentary mechanical logic can include a complimentary tab and slot that mate only when the pusher and the delivery cylinder are at certain angles relative to each other.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a fastener assembly according to an embodiment of the invention.

FIG. 2 depicts an individual fastener according to an embodiment of the invention.

FIG. 3 depicts a fastener and suture combination, in accordance with an embodiment of the invention.

FIG. 4 depicts a fastener and delivery tube combination, in accordance with an embodiment of the invention.

FIG. 5 depicts a delivery system for fastener assembly insertion in accordance with an embodiment of the invention.

FIG. 6 depicts an anchor tube and pusher tube for ejecting cylindrical fasteners in accordance with an embodiment of the invention.

FIG. 7 depicts a delivery system assembly in accordance with an embodiment of the invention.

FIG. 8 depicts a cross-sectional view of a delivery system assembly in accordance with an embodiment of the invention.

FIG. 9 depicts an anchor tube assembly in accordance with an embodiment of the invention.

FIG. 10 depicts a pusher tube assembly in accordance with an embodiment of the invention.

FIG. 11 depicts an obturator tube assembly in accordance with an embodiment of the invention.

FIG. 12 depicts an alternative delivery tube assembly in accordance with an embodiment of the invention.

FIG. 13 depicts a peg and slot mechanical logic system in accordance with an embodiment of the invention.

FIGS. 14 and 15 depict processes for inserting the fastener assembly, in accordance with embodiments of the invention.

FIG. 16 depicts a fastener assembly deployment test bed in accordance with an embodiment of the invention.

FIG. 17 depicts pull-out test results in accordance with an embodiment of the invention. In this plot, the anchoring device is labeled “UMA”.

FIG. 18 depicts insertion of a fastener assembly in accordance with an embodiment of the invention.

FIG. 19 depicts an ultrasound image of insertion of a fastener assembly in accordance with an embodiment of the invention.

FIGS. 20-24 depicts tissue results due to insertion of a fastener assembly in accordance with embodiments of the invention.

FIG. 25 depicts the use of two fastener assemblies to close a gap created by a surgical instrument according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

In certain aspects, the invention provides a novel fastener assembly for securing a first bodily wall to a second bodily wall. In other aspects, the invention provides for an anchoring device which inserts the fastener assembly inside the body of a subject. In yet other aspects, the invention provides a method of securing a first bodily wall to a second bodily wall using the fastener assembly and implements of the invention.

### Fastener Assembly

Sutures or fasteners are used during medical procedures to anchor one bodily wall to another bodily wall. Conventional suture or fastener insertion techniques result in large incisions and invasive procedures, thereby requiring long recovery periods and increased risk for resulting complications (e.g., secondary infections, etc.). Additionally, after such procedures, sutures are known to cause morbidity (e.g., ischemia) of the surrounding tissue and often cause pain and discomfort for the subject. Fasteners established in the art also cause discomfort due to their rigidity and the fact that they are often secured within one of the bodily walls.

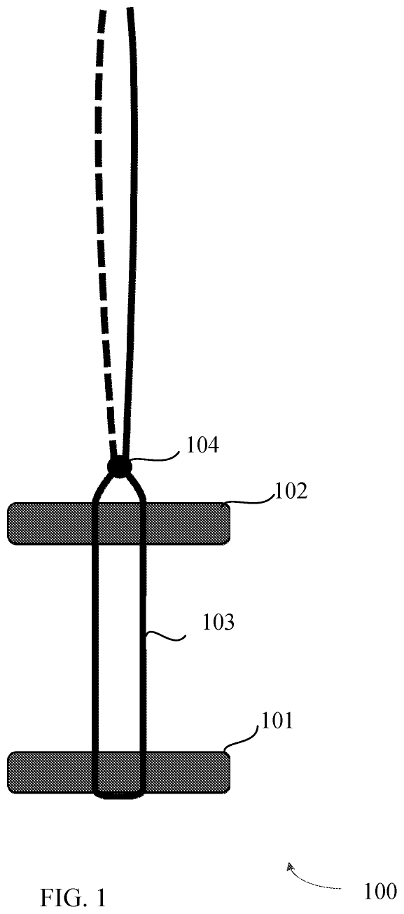

Referring to FIG. 1, one embodiment of the invention provides a novel fastener assembly 100 comprising a first fastener 101, a second fastener 102, and a string 103 connecting the first fastener 101 with the second fastener 102. The ends of the string may be connected through a locking-sliding knot 104 (e.g., a modified Meltzer knot, a Weston knot, etc.) so that when tightened, the first fastener 101 and the second fastener 102 are pulled towards one another.

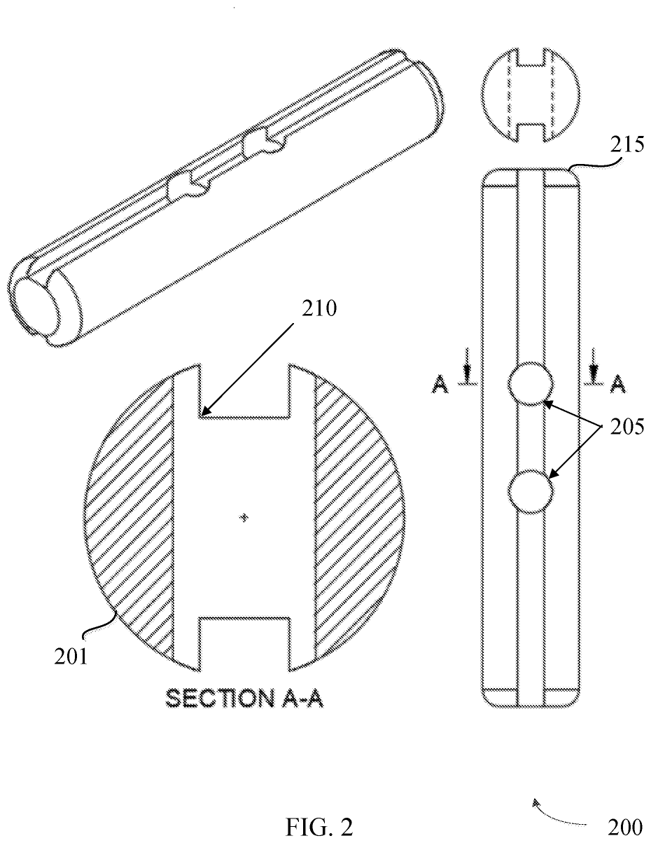

FIG. 2 provides various views 200 of a fastener 201, such as first fastener 101 or second fastener 102 with reference to FIG. 1. The fastener 201 may be designed with a high length-to-diameter ratio. This high length-to-diameter ratio may aid the fastener 201 in rotating to position itself flat against a bodily wall. Fastener 201 may include lumens 205 (e.g., holes). Sutures may be passed through one or each of the lumens 205. Lumens 205 may allow for a suture to aid in the rotation of the fastener 201 when the suture is pulled.

The fastener 201 may also include a set of slots 210 that run along the longitudinal axis of the fastener 201. The sutures 103 may lie in the slots 210 when the fastener assembly resides in the delivery tube (e.g., fastener 201 is in a perpendicular position relative to a bodily wall). Constraining the suture 103 to follow a single path may prevent unwanted migration of the suture 103 between the fastener 201 and the walls of the delivery tube, which may result in high resistance to the movement of the fastener assembly in the delivery tube and possible jamming.

Fastener 201 may additionally or alternatively include filleted ends 215 to provide for atraumatic surfaces when the fastener 201 is present in a body (e.g., a subject). The radius of the filleted portion may be designed to remove traumatic edges from the fastener 201 (e.g., on the scale of 100 microns).

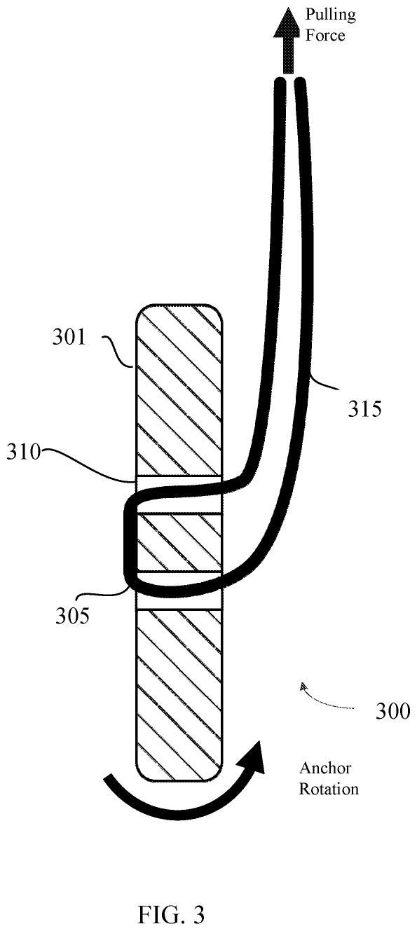

FIG. 3 illustrates a fastener and suture combination 300, such as fasteners and sutures described with reference to FIGS. 1 and 2. The fastener 301 may include lumens 305 and 310. The suture 315 may be passed through the lumens 305 and 310 in a loop configuration, where both ends of the suture exit the same side of the fastener 301. When a pulling force is applied to the ends of the suture, the loop configuration around the fastener 301 forces the fastener 301 to rotate from a substantially longitudinal position (e.g., parallel to an axis along the length of the delivery tube) to a substantially latitudinal position (e.g., perpendicular to the axis along the length of the delivery tube).

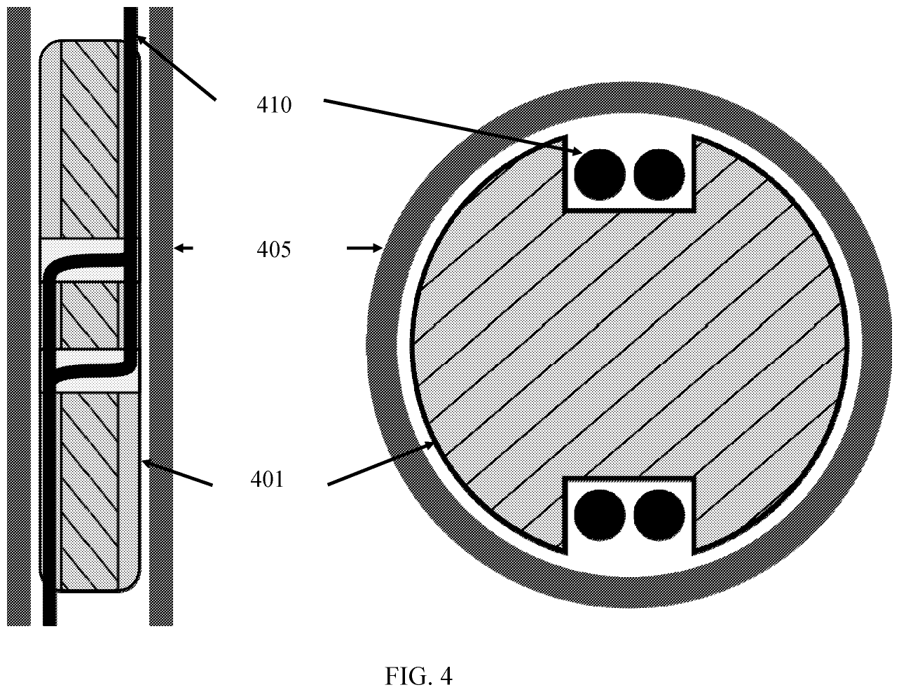

FIG. 4 illustrates a fastener and delivery tube combination 400. The fastener 401 is located within the walls of the delivery tube 405. The suture 410 runs along the longitudinal slots of the fastener 401 and through the lumens of the fastener 401. Here, fastener 401 is the fastener designed to rest exterior to the first bodily wall, with a second fastener (not shown) designed to rest interior to a second bodily wall. In this situation, the suture 410 runs through and around the lumens of the fastener 401, connecting the fastener 401 to both the second fastener below as well as the slip knot from above.

### Fastener Dimensions

As described herein, a cylinder is defined as a surface consisting of each of the straight lines that are parallel, or substantially parallel, to a given straight line and pass through a given curve. Further, the dimensions of the cylindrical fastener and corresponding lumens may be designed based on the desired results of the pending surgery. For example, when conducting a procedure on a human subject, the cylindrical fastener may be designed with a smaller diameter as opposed to a fastener intended to be used for a procedure conducted on a horse. Furthermore, different shapes of the fastener (e.g., disk, sphere, rectangular prism, triangular prism, etc.) may be used based on the type of procedure used as well as the intended subject. In addition, the fastener may change shape following deployment from the delivery device (e.g. foldable disk, foldable rectangle, etc.).

In some cases, a custom shape may be designed (e.g., via a 3-D printer or the like) for a subject-specific cylindrical fastener. As such, the distance and location of the lumens may be determined based on the selected shape, dimensions, and desired results. In an exemplary embodiment, the distance between the lumens (e.g., measured from closest-edge-to-closest-edge) of a fastener is approximately 2 mm.

### Fastener and Suture Composition

In some cases, the fasteners and sutures are composed of resorbable material, which may biodegrade over a period of time. This may be beneficial in cases where the fasteners are to be used for temporary positioning. The fasteners, suture, or a combination thereof, may be composed of any biodegradable or resorbable polymer, including but not limited to: polyglycolide-lactide copolymers (e.g. polyglactin 910), polydioxanone (PDS), polyglycolic acid, polylactic acid, polycaprolactone, polylactide-caprolactone copolymers, thermoplastic bioresorbable polyurethanes with degradable hard and soft segments, synthesized from polyester polyols, and aromatic or aliphatic isocyanates.

In some of these cases where resorbable material is relied on, the composition may be selected based on a predetermined degradation time, where the structure of the fastener and/or suture fails after the predetermined degradation time (e.g., on the scale of 1 to 36 months). Alternatively, the composition for the fasteners and/or suture may be selected for its ability to maintain its structure for an extended period of time. For example, in cases where a ligament is to be approximated, a composition that maintains it structural integrity permanently may be selected.

The terms “string” and “suture” are used interchangeably herein. Either can be natural, synthetic, hypoallergenic, biocompatible, and/or absorbable. Exemplary materials include absorbables (e.g., polylactic acid, MONOCRYL® sutures available from Johnson & Johnson Corporation of New Brunswick, N.J., poly (4-hydroxybutyric acid) (P4HB available under the MONOMAX® trademark from Aesculap AG of Tuttlingen, Germany)), as well as non-absorbables (e.g., nylon, polyester, PVDF, and polypropylene).

### Bodily Wall Types

The bodily walls that are to be approximated by the fastener assembly is not to be limited to soft tissue of a subject. For example, some cases may include different type of material to be approximated, including but not limited to ligaments, bone, cartilage, organs, soft tissue, skin, connective tissue, and cavities surrounding different tissues.

### Delivery Device

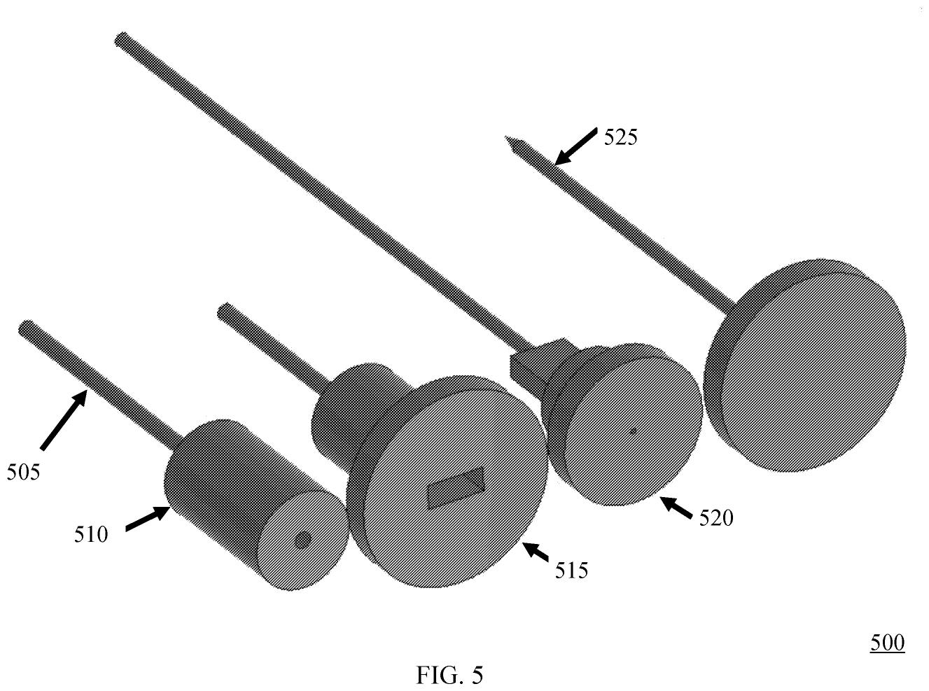

A delivery device may include concentric tubes that contain the fastener assembly. The delivery device may push the fastener assembly and advance the locking-sliding knot. An example of a delivery device 500 is illustrated in FIG. 5, where the delivery device may include a delivery tube 505, a mating housing 510, an anchor tube and handle 515, a pusher tube and handle 520, and an obturator 525.

### Delivery Tube

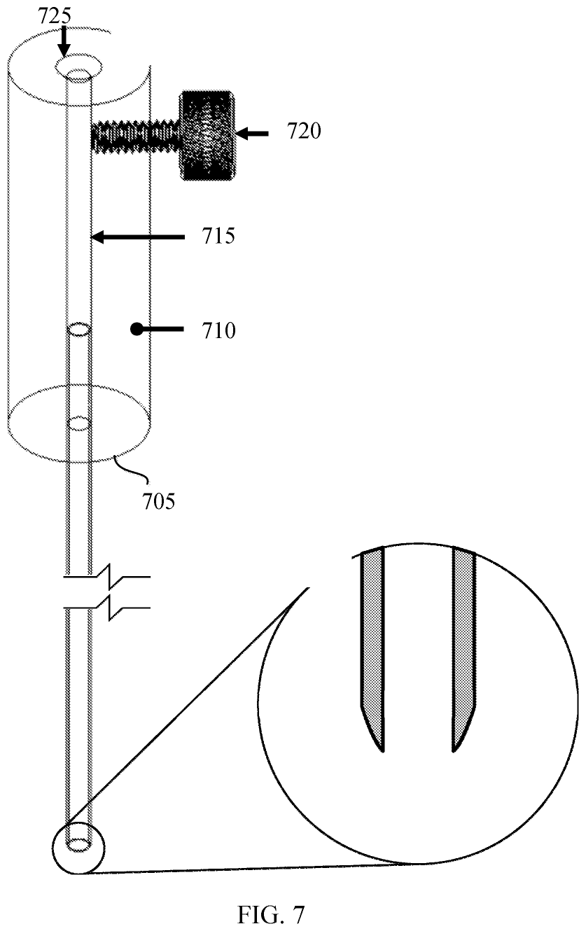

The delivery tube 505 may have an inner diameter that matches, or approximately matches, the diameter of the fasteners. The delivery tube 505 may be long enough to penetrate to the space within the innermost layer to be approximated. The delivery tube 505 may include a low wall thickness to keep the outer diameter low, which may minimize tissue damage. The long, straight tube of the delivery tube 505 may aid in visualization under medical imaging, such as ultrasound imaging. An example of a delivery tube is illustrated in FIG. 7. Here, delivery tube 700 includes a delivery tube 705, a mating housing 710, a mating hole 715, a locking mechanism 720, and an anchor tube entry port 725.

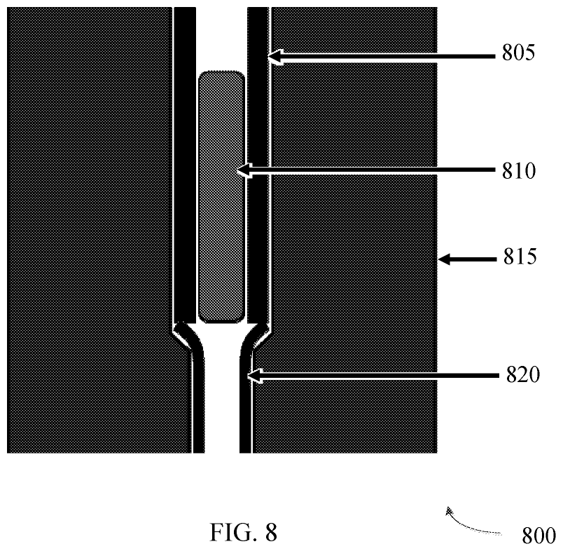

Referring to FIG. 5, the tip of the delivery tube 505 may be beveled so that when the obturator 525 is in place, there exists a smooth transition from the surface of the obturator 525 to the external surface of the delivery tube 505. The other end of the delivery tube 505 may be flared to facilitate smooth movement of the fastener from the anchor tube 515 to the delivery tube 505 during deployment. An example of this facilitation is illustrated by the cross-sectional view 800 of a delivery system assembly in FIG. 8, which includes an anchor tube 805, a fastener 810, a mating housing 815, and a delivery tube 820.

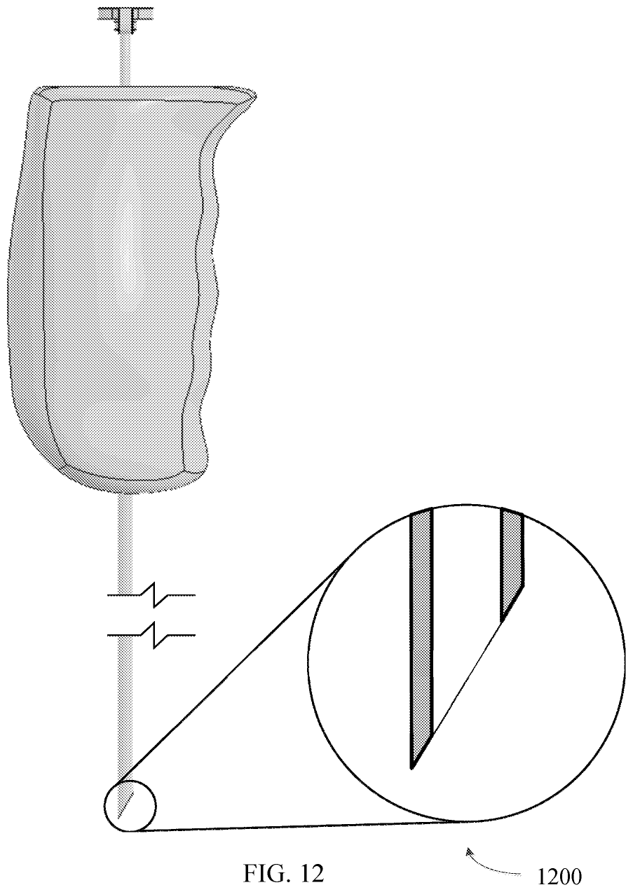

An alternative embodiment may include a delivery tube with a sharp tip. The sharp tip may be akin to that of a hypodermic needle. The first fastener, in this embodiment, may be used to occlude the lumen of the delivery tube to avoid coring out tissue during piercing. This embodiment may decrease travel distance for a pusher tube due to the fasteners being already present at the tip of the delivery tube at the initiation of actuation. Additionally, due to the shorter distance, the delivery tube may include alternate gripping methods, such as a hammer grip, with a thumb used to actuate the delivery tube. An example of this alternative embodiment is found in FIG. 12.

### Mating Housing

The delivery tube 505 may be permanently attached to the mating housing 510, which may include holes that bring the delivery tube 505 and the anchor tube 515 together concentrically when the anchor tube 515 is inserted. The mating housing 510 may also include a feature to hold the anchor tube 515 in place after mating (e.g., set screw, thumb screw, magnets, etc.). The opening of the mating housing 510 where the anchor tube 515 is inserted may include a bevel that may guide the anchor tube 515 into the hole. In some cases, the mating housing 510 is also permanently attached to the anchor tube 515, thereby permanently connecting the anchor tube 515 with the delivery tube 505.

### Anchor Tube

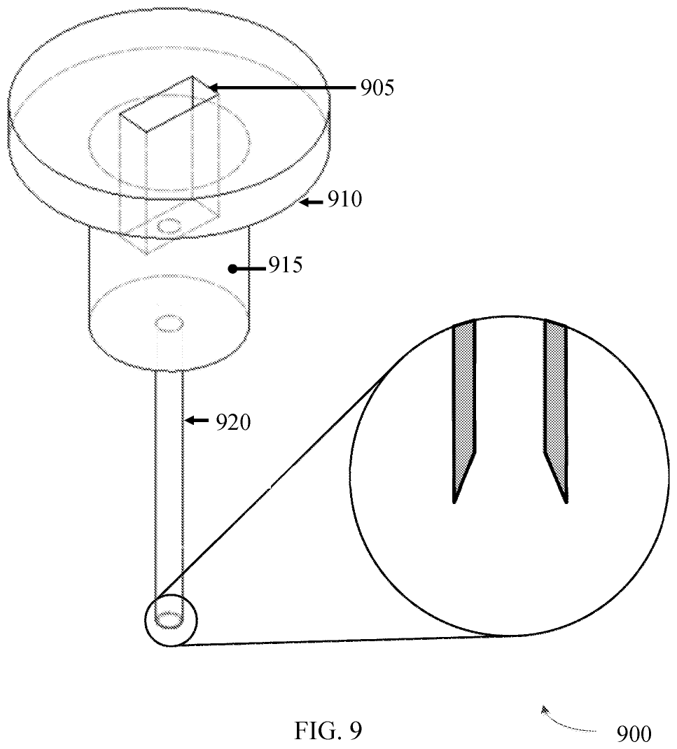

The anchor tube assembly may include an inner diameter that may match the diameter of the fasteners and may be long enough to contain the fastener assembly with the suture loop fully stretched out. The tip of the anchor tube 515 may include a bevel that may guide the fasteners into the anchor tube 515 during loading. The anchor tube 515 may be permanently attached to an anchor tube handle. The handle may include a hole that matches a peg on a pusher tube handle, such as pusher tube 520. The hole combined with the peg on the pusher tube handle may provide a mechanical logic that ejects the fasteners individually at the correct locations. The handle of the anchor tube 515 may allow the operator to grip the handle below a disc between two fingers and apply downward forces on the pusher tube handle. An example of an anchor tube assembly is illustrated in FIG. 9. Anchor tube assembly 900 includes a hole for a pusher tube peg 905, a gripping surface for an operator 910, an anchor tube handle 915, and an anchor tube 920.

### Pusher Tube

The pusher tube 520 may include an outer diameter that allows for slight clearance with the inner diameter of the anchor tube 515 and delivery tube 505. The clearance may allow for smooth motion but may be kept to a minimum to prevent the migration of the suture to any excess clearance space during ejection, which may result in increased resistance to motion and possible jamming. The tip of the pusher tube 520 may include an opening large enough to allow suture of a given size to pass freely through the opening. When the suture is pulled, the suture may pass through the pusher tube 520 freely but the knot may not advance through the opening. This may advance the knot down the suture, thereby tightening the fastener assembly. The opening at the tip may include a bevel, which may simplify feeding the suture during loading.

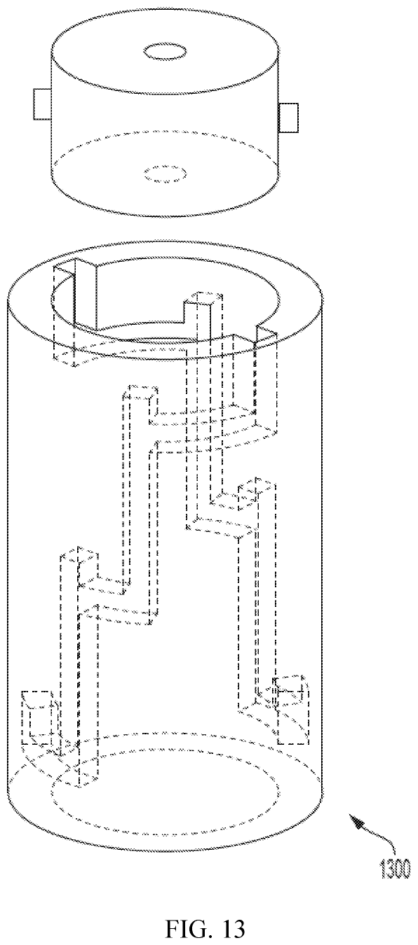

Additionally, the pusher tube 520 may be attached to a pusher tube handle. The pusher tube handle may include a peg that matches the hole of the anchor tube handle. The length of the peg may be greater than the length of a fastener. Furthermore, the length of the pusher 520 may be designed such that during operation, when the bottom of the peg contacts the top surface of the anchor tube handle, the first fastener is fully ejected and the second fastener begins its exit from the delivery tube 505. This may ensure that the fasteners are fully ejected at the desired locations. An example of a peg-and-hole mechanism is illustrated in FIG. 13. Alternatively, a pin and track mechanism may be used in lieu of the peg-and-hole mechanism 1200. The pin may slide into the track, which may include stops to allow single fastener ejection and locking of the pusher tube in a lowermost position for ease of use during knot advancement.

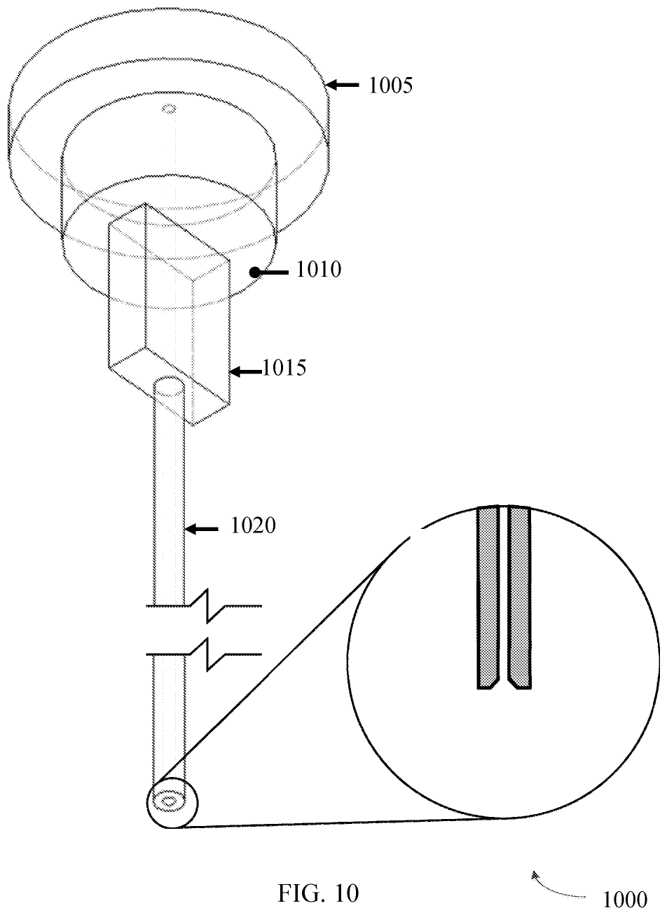

Additionally, the top of the pusher tube handle may be flat, which may provide sufficient room for the operator to push on the pusher tube handle with a thumb or a second hand. An example embodiment of the pusher tube assembly is illustrated in FIG. 10. Pusher tube assembly 1000 may include a gripping surface 1005, an anchor tube handle 1010, a peg for a hole 1015, and pusher tube 1020.

### Obturator

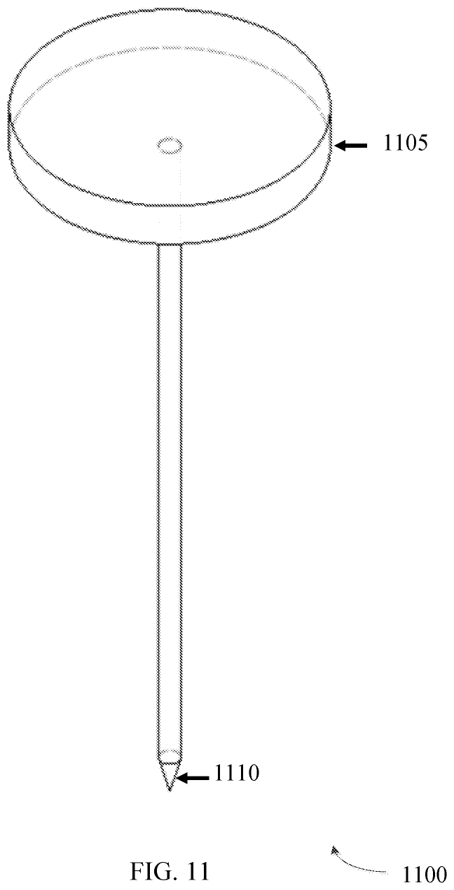

The obturator 525 may have a diameter that matches the inner diameter of the delivery tube 505, which may minimize a gap between the obturator 525 and the delivery tube 505. This may allow for a smooth surface transition from the obturator 525 to the delivery tube 505 as the fastener assembly slides through tissue. Additionally, the obturator, such as obturator 1100 in FIG. 11, may include a sharp-tipped end 1110 that may pierce tissue while causing minimal tissue damage. Additionally or alternatively, the obturator 1100 may be attached to a flat disc handle 1105. The length of the obturator may be such that when the handle lies against the face of the mating housing, the pointed tip of the obturator is just clear of the delivery tube. Referring to FIG. 5, the long and straight nature of the obturator 525 may aid in visualization under ultrasound imaging. Additionally or alternatively, the obturator 525 may be composed of metal for autoclave sterilizability. Furthermore, any one of the delivery assembly, the anchor assembly, the pusher assembly, or any combination thereof, may also be composed of metal for similar reasons.

### Camera or Endoscope Addition

Some embodiments may allow for a camera or endoscope to be implemented in conjunction with the delivery system. For example, a digital camera may be placed at the tip of the instrument. Additionally or alternatively, there may be a channel inside of the instrument that may allow passage of an endoscope (e.g., a 1 mm endoscope) either to the end of the device tip or past the device tip. The camera or endoscope may allow for an expanded field of view (e.g., 30 or 40 degrees) beyond the tip of the device. In addition, a camera may also be inserted through a previously placed port (e.g., a plastic port), such as ports used in fetal surgery procedures to confirm the proper placement of the anchoring device. Alternatively, the device may be inserted using a sharp trocar placed down the barrel of the device and removed once the device is in position.

### Insertion Process

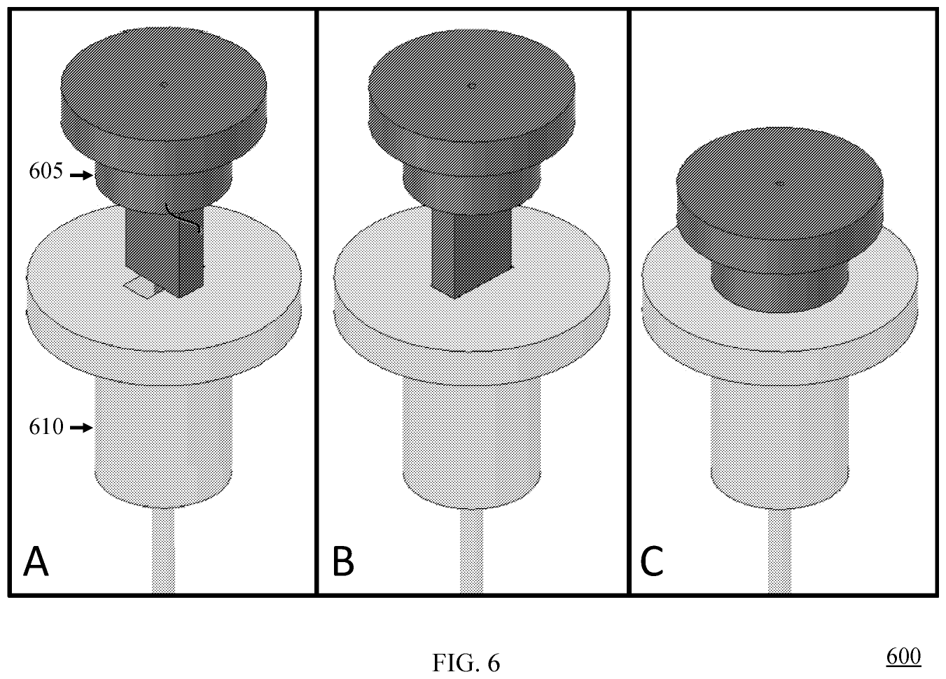

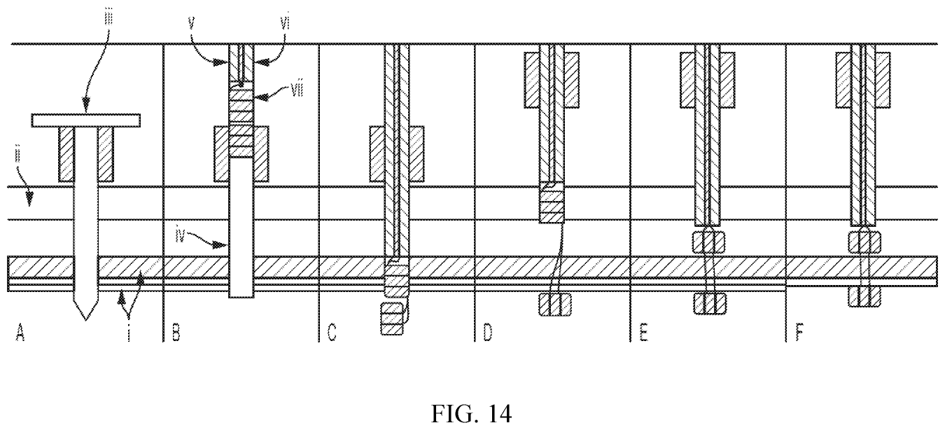

FIGS. 14 illustrates an exemplary insertion process of the fastener assembly. The obturator may be mated with the delivery tube. In Step A of FIG. 14, the obturator may be used to penetrate tissue of a subject through to the space inside the innermost layer. The obturator may then be removed, leaving the tip of the delivery tube inside the innermost layer to be approximated. A pusher tube may be inserted partially into an anchor tube and the ends of the suture may be threaded through the pusher tube. The fasteners may be loaded into the anchor tube. The fasteners may be preloaded into the anchor tube or a cartridge at time of manufacture. At Step B of FIG. 14, the anchor and string may then be mated with the delivery tube in a mating housing, and may be locked in place. At Step C of FIG. 14, the pusher may then eject the first fastener by sliding down until the bottom of a peg of the pusher tube handle contacts the top surface of the anchor tube handle (e.g., as shown in Step A of FIG. 6). At Step D of FIG. 14, the delivery tube may then be retracted above the two layers to be approximated. The peg of the pusher tube may then be aligned with a hole on the anchor tube handle (e.g., Step B of FIG. 6) and, at Step E of FIG. 14, the pusher tube may then be pushed in completely to eject the second fastener (e.g., Step C of FIG. 6). At Step F of FIG. 14, the ends of the suture may then be pulled, which may advance the locking-sliding knot, thereby approximating the layers. The pusher may then be removed, and the ends of the suture trimmed. Alternatively, in some cases the delivery tube may not be completely retracted from the two layers and may instead eject the second fastener prior to complete retraction. In these cases, the second fastener may be assisted into position above the first bodily wall (e.g., via CO₂ gas or by lifting or “tenting” the tissue with graspers).

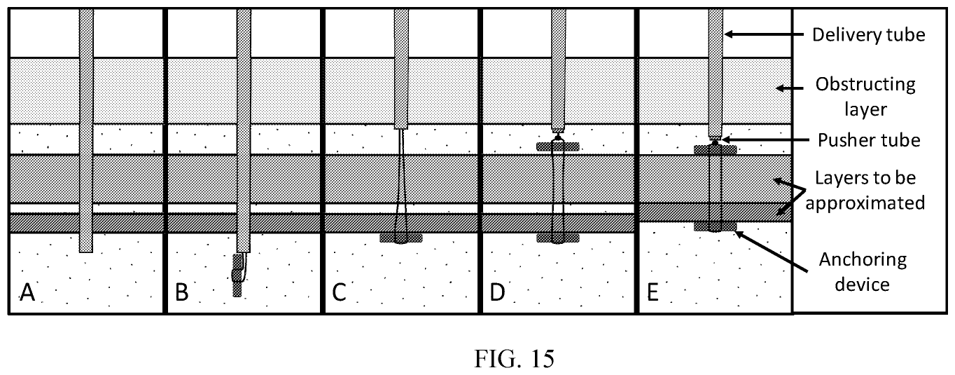

Another example of the insertion process is illustrated in FIG. 15. At Step A of FIG. 15, the delivery tube may be positioned through the first and second bodily walls. At Step B of FIG. 15, a first fastener may be ejected below the second bodily wall. At Step C of FIG. 15, the delivery tube may be retracted above the second and first bodily walls. At Step D of FIG. 15, the second fastener may be ejected from the delivery tube. At Step E of FIG. 15, the fastener assembly may be tightened, thereby approximating the first and second bodily walls.

FIG. 6 illustrates an anchor tube and pusher tube combination while in different stages during the insertion process. At Step A of FIG. 6, the pusher assembly ejects the first fastener by sliding down until the bottom of the peg of the pusher tube handle 605 contacts the top surface of the anchor tube handle 610. At Step B of FIG. 6, the peg is aligned with the hole on the anchor tube handle 610. At Step C of FIG. 6, the pusher tube is pushed all the way to eject the second fastener.

### 1st Example of Fastener Deployment

Many fetal surgeries that are performed percutaneously utilize a vascular access port (e.g., 9-12 Fr vascular access port), which may be placed into a uterus under ultrasound guidance through which an endoscope is placed. In order to anchor the chorioamniotic membranes prior to vascular port placement, two fastener assemblies may be deployed under ultrasound guidance inside the uterus and the chorioamniotic membranes. While holding these two fastener assemblies with the delivery tubes, the vascular port may be placed in between the delivery tubes under ultrasound guidance through which the surgical operative procedure will be performed. Once the surgical procedure is completed, the operative fetoscope may then be withdrawn, leaving the barrel of each delivery tube partially embedded within the uterine wall with the first fasteners deployed inside the amniotic cavity.

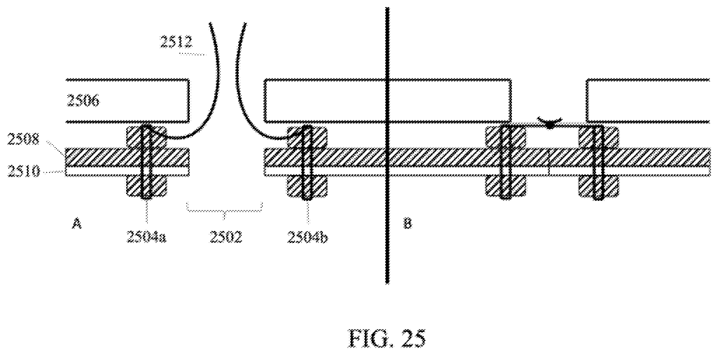

The abdominal wall may then be elevated with towel clamps such that the abdominal wall is pulled upward toward the ceiling, which may provide a space between the uterus and inside of the abdominal wall. The vascular port may be withdrawn into this space through which a scope may be placed. The second fasteners may then be deployed and tightened under direct visualization to secure the membranes against the outside of the uterine surface using a sliding knot. Direct visualization with the scope may also assist in preventing uterine blanching from occurring, which may suggest that the knot is too tight. The suture may then be cut under vision (e.g., via scissors, etc.) placed down the vascular port. The delivery tubes may then be removed from the subject. The suture tails of the two anchoring devices may be used to close the hole made by the vascular port following fetal surgery using sliding knots, as shown in FIG. 25.

### 2nd Example of Fastener Deployment

Some fetal surgeries may require the fixation of the membranes prior to placing an access port. In this circumstance, the delivery tube with a camera or endoscope (e.g., a STORZ™ 1 mm endoscope) may be used as a standalone instrument. Initial access to the abdominal cavity may be provided using a standard laparoscopic entry (e.g., a 5 mm) port. Once the abdomen is entered, gas (e.g., CO2) may be introduced to create space between the uterus and the abdominal wall. Alternatively, graspers may be used to tent the abdomen.

A sharp-tipped (e.g., diamond-tipped) trocar may be placed down the barrel of the delivery tube. The anchor delivery device may then be pushed through the abdominal wall via the entry port. Once inside the abdomen, the anchor delivery device may be pushed through the uterine wall under vision or ultrasound to enter the amniotic cavity. The trocar may then be removed and the anchor system may be loaded into the barrel of the delivery tube.

The first fastener may be deployed under vision inside the uterus and inside the membranes. The delivery tube barrel may be removed from the inside the uterus back into the abdominal cavity, thereby leaving the first fastener in the amniotic cavity. The second fastener may then be deployed and tightened under vision. Afterwards, the suture may be cut and removed from the delivery tube.

The delivery tube may then be reloaded with the trocar and the same process may be repeated to enter the uterus in a different location. The trocar may be removed once the delivery tube is in the amniotic fluid and the set of fasteners may be loaded and deployed as discussed above. This process may be repeated as necessary to place the desired or required number of fasteners inside the uterus. Once sufficient fasteners are placed, the barrel may then be withdrawn through the entry port. The suture tails of the two anchoring devices may be used to close the hole made by the trocar following fetal surgery using sliding knots, as shown in FIG. 25.

### Benchtop Pull-Out Force Characterization

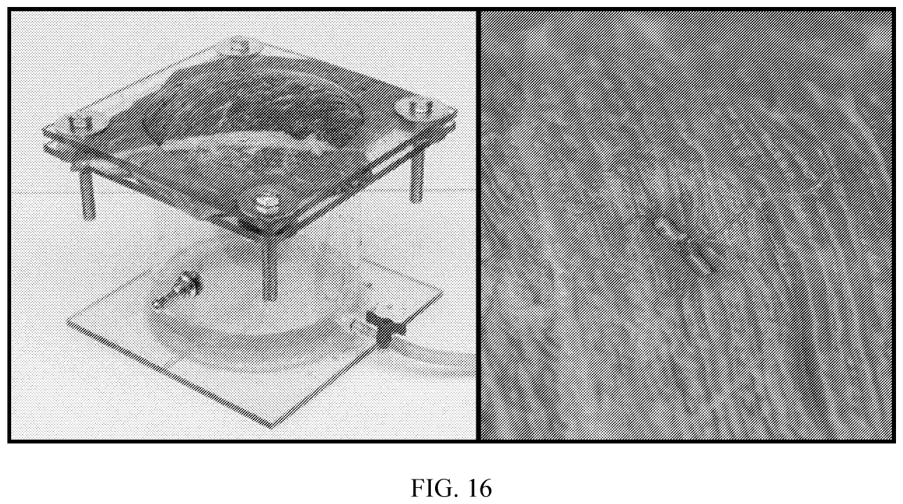

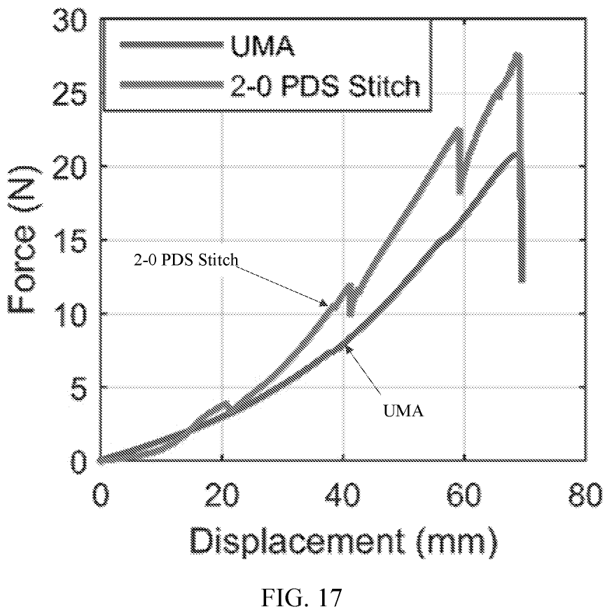

To compare the device to the current surgical standard, pull-out tests were performed on a test bed designed for testing these devices. As shown in FIG. 16, the test bed includes a sheet of muscle tissue clamped over the opening of a sealed chamber which may then be pressurized to simulate the stiffness of the target tissue. A pair of fasteners was deployed in a sheet of bovine skeletal tissue on the test bed. The top fastener was then attached to the jaws of an MTS mechanical testing frame. The jaws were retracted at a rate of 10 mm/minute until the fasteners pulled free from the tissue. Pulling force was measured using a 500 Newton load cell sampled at 500 Hz. Peak pull-out force was measured. A single stitch was placed on the same tissue sample using a two-handed surgical tie with 2-0 PDS suture and SH needle. The suture loop was attached to the jaws of the testing frame with a hook and the jaws were retracted at 10 mm/minute. As shown in FIG. 17, peak pull-out force was found to be 21 Newtons for the anchoring device (labeled “UMA”) and 28 Newtons for the single stitch.

Thus, the fastening system displays holding strength comparable to the currently used stitching method while also allowing surgery to be performed fetoscopically.

### Acute Handling Characterization

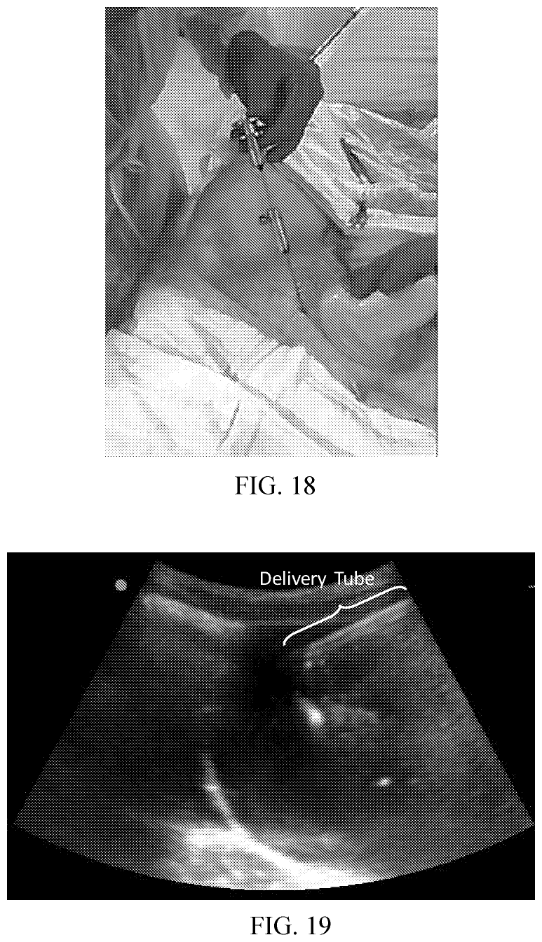

To test acute handling characteristics of the device and the feasibility of full percutaneous delivery, deployment was tested in a porcine cadaver model (e.g., shown in FIG. 18) using the urinary bladder as an analogue to a pregnant uterus. Under ultrasound guidance, three devices were successfully deployed in the bladder fully percutaneously. The delivery device was seen to be clearly visible under ultrasound imaging, as seen in FIG. 19. Additionally, in a chronic sheep study, 10 devices were successfully implanted across 5 sheep uteri and explanted after 30 days.

### Host Response Characterization

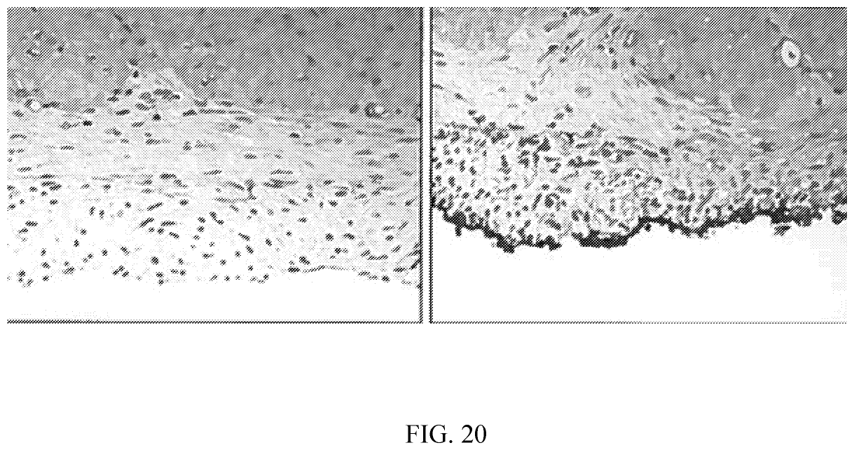

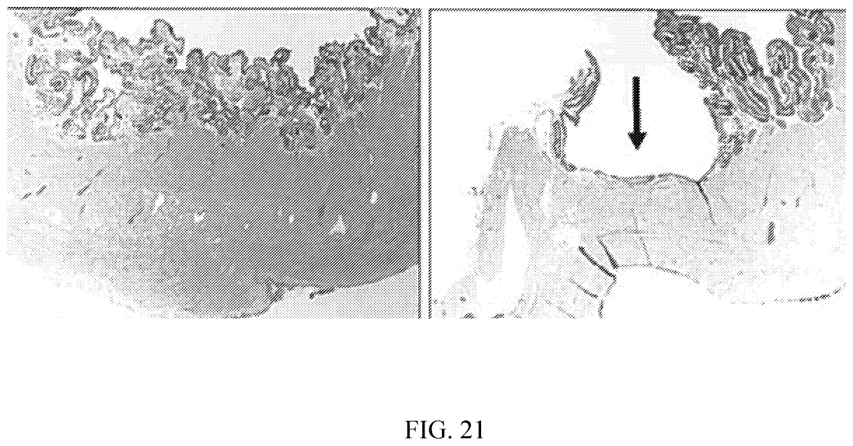

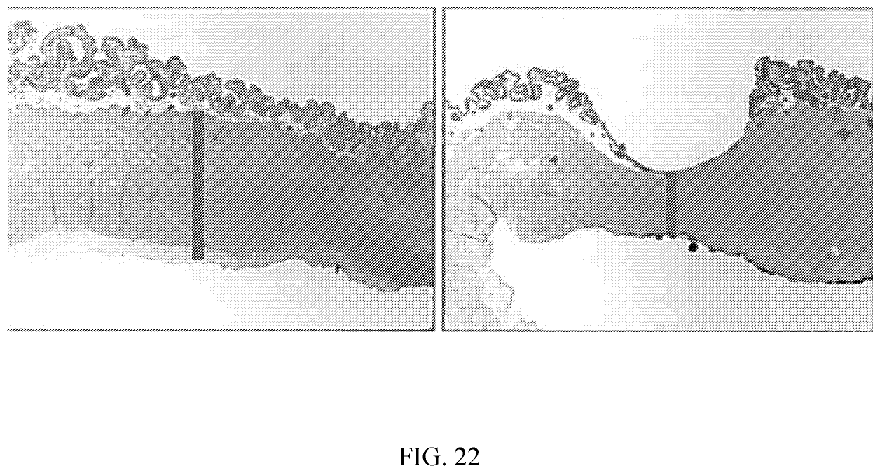

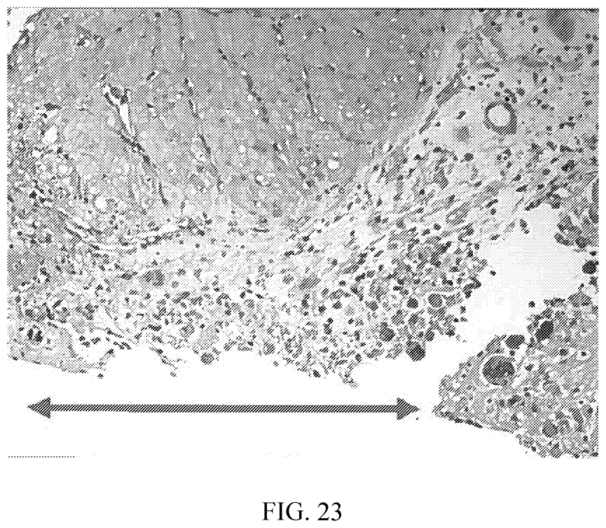

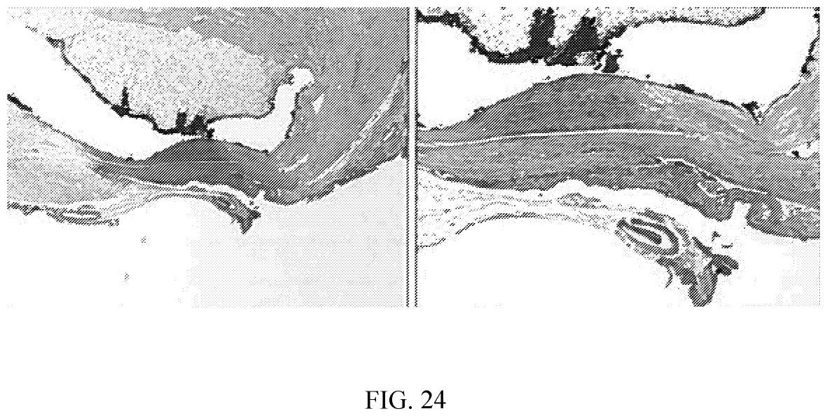

To measure the host response to the device while it is implanted, fasteners were implanted in the uteri of pregnant rabbits in their third trimester. Host response was examined through histology of sections of the uterus at the implant site, as shown in FIGS. 20-24. The data showed that the implant was well tolerated over a 7-day period with mild to moderate inflammation similar to the response elicited by currently available sutures used for incision closure. Some attenuation of endometrial glands and wall thickness was observed. We demonstrated the functionality of the anchor deployment system under ultrasound guidance in the rabbit chronic model. Of note, no miscarriage or fetal deaths occurred while the chorioamniotic anchors remained in place. Out of the six rabbit experiments, in one instance histological evidence of tissue necrosis was noted most likely because of the mechanical effect of an anchor where the sliding knot was too tight.

### Use to Close Gap

Referring now to FIG. 25, the fastener assemblies described herein can be utilized to close a gap 2502 (e.g., created by a surgical instrument such as a trocar punctured through the abdominal wall 2506, uterine wall 2508, and chorioamniotic membranes 2510). Two or more fastener assemblies 2504a, 2504b are implanted around gap 2502 through the uterine wall 2508 and chorioamniotic membranes 2510. The trailing suture ends 2512 from each fastener assembly 2504a, 2504b can be tied together to pull the walls of gap 2502 together and close the gap.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A fastener assembly for securing a first bodily wall to a second bodily wall,
the fastener
assembly comprising:
a first cylindrical fastener defining a first set of cross lumens, each of the first set of cross lumens extends substantially perpendicular to a central axis of the first cylindrical fastener;
a second cylindrical fastener defining a second set of cross lumens, each of the second set of cross lumens extends substantially perpendicular to a central axis of the second cylindrical fastener; and
a string running through the first set of cross lumens of the first cylindrical fastener, and the second set of cross lumens of the second cylindrical fastener, such that the tightening of the string will draw the first cylindrical fastener and the second cylindrical fastener towards each other.

2. The fastener assembly of claim 1, wherein the string is fastened to itself with a locking-sliding knot.

3. The fastener assembly of claim 1, wherein the string is fastened to itself with a Weston knot or a Meltzer knot.

4. The fastener assembly of claim 1, wherein the first cylindrical fastener and the second cylindrical fastener each have a maximum cross-sectional dimension of about 2.5 mm.

5. The fastener assembly of claim 1, wherein the first cylindrical fastener, second cylindrical fastener, and the string comprise a resorbable polymer composition.

6. The fastener assembly of claim 5, wherein the resorbable polymer composition comprises one or more materials selected from the group consisting of polyglycolide-lactide copolymers, polyglactin 910, polydioxanone (PDS), polyglycolic acid, polylactic acid, polycaprolactone, and polylactide-caprolactone copolymers.

7. The fastener assembly of claim 5, wherein the first cylindrical fastener, the second cylindrical fastener, the string, of a combination thereof, comprises one or more materials selected from the group of thermoplastic bioresorbable polyurethanes with degradable hard and soft segments, synthesized from polyester polyols, and aromatic or aliphatic isocyanates.

8. The fastener assembly of claim 5, wherein the design of the cylindrical fastener and string, and the polymer composition are engineered to provide a target degradation time.

9. The fastener assembly of claim 1, wherein the first cylindrical fastener further defines a recess located along a longitudinal axis of the first cylindrical fastener.

10. The fastener assembly of claim 9, wherein a portion of the string rests in the recess when the longitudinal axis of the first cylindrical fastener is perpendicular to the first bodily wall, the second bodily wall, or both.

11. The fastener assembly of claim 9, wherein the longitudinal axis of the first cylindrical fastener repositions from being substantially perpendicular to the first bodily wall, the second bodily wall, or both, to being substantially parallel to the first bodily wall, the second bodily wall, or both, during the tightening of the string.

12. An anchor delivery or deployment device comprising:
a shaft defining a longitudinal cavity, the longitudinal cavity having a set of dimensions to fit the fastener assembly of claim 1 within the longitudinal cavity.

13. The anchor device of claim 12, further comprising an obturator having a piercing end, the obturator having a width to allow the obturator to fit within the longitudinal cavity of the shaft.

14. The anchor device of claim 13, wherein the obturator terminates in a handle opposite the piercing end.

15. The anchor device of claim 12, wherein the shaft further comprises a cannula.

16. A method of securing a first bodily wall to a second bodily wall of a subject, the method comprising:
inserting the shaft of the anchor device of claim 12 into the skin of the subject, through the first bodily wall and through the second bodily wall;
inserting the fastener assembly into the shaft of the anchor device;
deploying the first cylindrical fastener inside of the second bodily wall;
withdrawing the shaft of the anchoring device through the second bodily wall and the first bodily wall;
deploying the second cylindrical fastener from the shaft of the anchoring device and outside of the first bodily wall;
cinching the first cylindrical fastener and the second cylindrical fastener together by pulling one or both ends of the string; and
withdrawing the shaft of the anchoring device completely from the skin of the subject.

17. The method of claim 16, further comprising:
inserting an obturator into the skin of the subject to define an opening through the first bodily wall and the second bodily wall, wherein inserting the shaft of the anchor device further comprises inserting the shaft of the anchor device into the opening.

18. The method of claim 17, wherein insertion and manipulation of the obturator is aided by medical imaging.

19. The method of claim 18, wherein the medical imaging is ultrasound or direct visualization using a laparoscope or fetoscope.

20. The method of claim 16, wherein the first bodily wall is a uterine wall and the second bodily wall is a placental membrane.

21. The method of claim 16, wherein the subject is a mammal.

22. The method of claim 16, wherein the subject is a human.

23. The method of claim 16, wherein the subject is a pregnant female.

24. A kit comprising:
a fastener assembly; and
instructions to perform the method of claim 16.

25. A fastener assembly for securing a first bodily wall to a second bodily wall,
the fastener
assembly comprising:
a first cylindrical fastener defining:
a first axial external channel;
a second axial external channel;
a first cross lumen extending between the first axial external channel and the second axial external channel; and
a second cross lumen extending between the first axial external channel and the second axial external channel;
a second cylindrical fastener defining:
a third axial external channel;
a fourth axial external channel;
a third cross lumen extending between the third axial external channel and the fourth axial external channel; and
a fourth cross lumen extending between the third axial external channel and the fourth axial external channel; and
a string running through the first cross lumen and the second cross lumen of the first cylindrical fastener, and the third cross lumen and the fourth cross lumen of the second cylindrical fastener, such that the tightening of the string will draw the first cylindrical fastener and the second cylindrical fastener towards each other.

26. A fastener system comprising:
a delivery cylinder comprising:
a proximal end adapted and configured for manipulation by a user; and
a distal end adapted and configured for advancement through one or more bodily walls;
a fastener assembly of claim 1; and
a pusher comprising:
a proximal end adapted and configured for manipulation by a user; and
a distal end adapted and configured for advancement into the delivery chamber, thereby advancing the fastener assembly;
wherein the proximal end of the delivery cylinder and the proximal end of the pusher have complimentary mechanical logic that:
limits initial advancement of the pusher rod to initially advance only a distal fastener beyond the distal end of the delivery cylinder; and
permits further advancement of the pusher in response to a second action to advance a proximal fastener beyond the distal end of the delivery cylinder.

27. The fastener system of claim 26, wherein the pusher defines an axial channel extending between the proximal end and the distal end, the axial channel adapted and configured to receive the string.

28. The fastener system of claim 27, wherein the axial channel is internal or external to the pusher.

29. The fastener system of claim 26, wherein the complimentary mechanical logic comprise a complimentary tab and slot that mate only when the pusher and the delivery cylinder are at certain angles relative to each other.
